(19) United States
(12) Patent Application Publication
(10) Pub. No.: US 2009/0105238 A1
(43) Pub. Date: Apr. 23, 2009

(54) Compounds and Relative use for the Control of Phytopathogens

(75) Inventors: Lucio Filippini, Novara (IT); Marilena Gusmeroli, Monza-Milano (IT); Silvia Mormile, Novara (IT); Gregorio Valea, Novara (IT); Luigi Mirenna, Milano (IT); Maria Gabriella Colucci, Pozzuoli-Napoli (IT); Fabio Apone, Napoli (IT)

Correspondence Address: James v Costigan;Hedman & Costigan, P.C., 1185 Avenue of the Americas, New York, NY, 10036, US

(73) Assignee: ISAGRO RICERCA S.r.l, Milano (IT)

(21) Appl. No.: 12/083,393

(22) PCT Filed: Oct. 10, 2006
(86) PCT No.: PCT/EP2006/009813

§ 371 Date: Apr. 28, 2008

(30) Foreign Application Priority Data
Oct. 17, 2005 (IT) MI2005A 001957
Dec. 22, 2005 (IT) MI2005A 002460

(51) Int. Cl. A01N 43/86; C07C 229/00; A01N 43/60; A61K 31/225

(52) U.S. Cl. 514/231.2; 560/171; 514/547; 514/252.12

(57) ABSTRACT

Amphoteric compounds are described, having a zwitterionic structure of the betainic type having general formula (I)

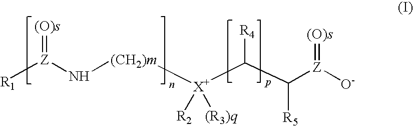

and their use for the control of phytopathogen fungi and/or the mitigation of abiotic and biotic stress.

The present invention relates to compounds and the relative use thereof for the control of phytopathogens.

Amphoteric surface-active agents, such as alkyl betaine, alkylamide alkyl betaine, hydroxysulfobetaine, are compounds which are known for their foaming, viscosizing, antistatic, softening properties, and thanks to their excellent affinity with other types of surface-active agents and intrinsic low irritating capacity with respect to the skin and eyes, are widely used in detergents and cosmetics.

It is also known that the above amphoteric surface-active agents can be used as components in formulations of agro-drugs as described, for example, in WO-A-97/47196 and EP-B-0597488 and in numerous other patents.

EP-A2-1542023, moreover, claims the use of amphoteric surface-active agents as “bioactivators” of agro-drugs already on the market, in suitable agronomic applications. In particular, their mixing with a herbicidal compound, such as for example, Glyphosate, improves its biological activity. It should be pointed out that the effect of “bioactivators” is exerted in an increased absorbability of the agrochemical active principle (herbicide, fungicide, insecticide, acaricide . . . ) inside the tissues of the plant or surface of the pathogen, or in an increased availability of the agrochemical active principle for the organisms of interest.

The compositions described in EP-A2-1542023 therefore allow a reduction in the applied concentrations of the active principles thus added.

In EP-A2-1542023, the amphoteric surface-active agents consequently merely act as a carrier of the active principles with which they are simply mixed, according to the logical role of a formulation component. A biological activity of the above surface-active agents is expressly excluded.

In the agronomical field, moreover, it is known that glycine betaine, when administered to fruit plants, contributes towards controlling abiotic and nutritional growth stress, reducing imperfections in the fruit peel and the tendency of the peel to break when ripening, as described in EP-A-0806897, acting as an osmolyte regulator.

The Applicant has now surprisingly found various amphoteric compounds which have a surprising activity in the agronomical field, as fungicidal and bactericidal products and which allow a prolonged protective action to be obtained on plants with respect to phytopathogen fungi and bacteria.

An object of the present invention is therefore an amphoteric compound characterized by a zwitterionic structure of the betainic type having general formula (I),

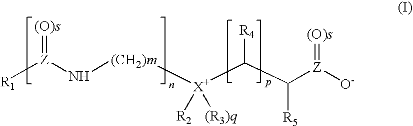

wherein:
R₁ represents a linear or branched C₁-C₂₆ alkyl group optionally substituted; a linear or branched C₁-C₂₆ haloalkyl group optionally substituted; a linear or branched C₁-C₂₆ alkoxyl group optionally substituted; a linear or branched C₁-C₂₆ alkylthio group optionally substituted; a linear or branched C₂-C₂₆ alkenyl group optionally substituted; a linear or branched C₂-C₂₆ alkinyl group optionally substituted; a C₃-C₃₀ cycloalkyl group optionally condensed or a condensed C₁₋₇ cycloalkyl group of the steroid type, optionally substituted; a C₃-C₃₀ cyclo-alkoxyl group optionally condensed and optionally substituted; a heterocyclic group optionally substituted; an aryl group optionally substituted; a hetero-aryl group optionally substituted; a linear or cyclic C₆-C₁₂ group of the saccharide type optionally substituted; a C₁-C₂₆ alkylamine group or a C₂-C₂₆ dialkylamine optionally substituted for n different from 0;
R₂ and R₃, the same or different, represent a C₁-C₃ alkyl group optionally substituted;
R₄ and R₅, the same or different, represent a hydrogen atom, or a linear or branched C₁-C₆ alkyl group optionally substituted; a linear or branched C₂-C₆ alkenyl group optionally substituted; a C₃-C₆ cycloalkyl group optionally substituted; a hydroxyl group; an aryl group optionally substituted; a hetero-aryl group optionally substituted; a heterocyclic group optionally substituted;
R₄ and R₅ can individually form a cycle together with R₂;
X represents a nitrogen or sulfur atom;
Z represents a carbon or sulfur atom;
m represents a number ranging from 1 to 5;
n and p represent a number ranging from 0 to 3;
q has the value of 0 for X=sulfur or the value of 1 for X=nitrogen;
s has the value of 1 for Z=carbon or the value of 2 for Z=sulfur.

The Applicant has also found that the compounds having general formula (I), in addition to having a direct fungicidal and bactericidal action, are capable of stimulating the natural defense systems of plants and inducing resistance in the plant itself; this method for controlling diseases and mitigating abiotic stress (temperature, salinity, drought, etc.) and biotic stress, is becoming of increasing interest, as it is based on the amplification of a natural process already present in the plant by the application of these compounds.

The Applicant has also surprisingly found that these compounds having general formula (I) represent an optimum form for controlling phytopathogens also in genetically modified vegetable varieties for amplifying the original natural defense.

A further object of the present invention therefore relates to the use of amphoteric compounds having a zwitterionic structure of the betainic type having general formula (I):

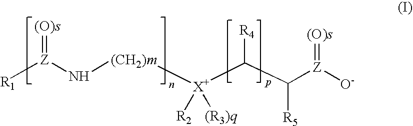

wherein:
R₁ represents a linear or branched C₁-C₂₆ alkyl group optionally substituted; a linear or branched C₁-C₂₆ haloalkyl group optionally substituted; a linear or branched C₁-C₂₆ alkoxyl group optionally substituted; a linear or branched C₁-C₂₆ alkylthio group optionally substituted; a linear or branched C₂-C₂₆ alkenyl group optionally substituted; a linear or branched C₂-C₂₆ alkinyl group optionally substituted; a C₃-C₃₀ cycloalkyl group optionally condensed or a condensed C₁₋₇ cycloalkyl group of the steroid type optionally substituted; a C₃-C₃₀ cyclo-alkoxyl group optionally condensed and optionally substituted; a heterocyclic group optionally substituted; an aryl group optionally substituted; a hetero-aryl group optionally substituted; a linear or cyclic C₆-C₁₂ group of the saccharide type optionally substituted; a C₁-C₂₆ alkylamine group or a C₂-C₂₆ dialkylamine optionally substituted for n different from 0;
R₂ and R₃, the same or different, represent a C₁-C₃ alkyl group optionally substituted;
R₄ and R₅, the same or different, represent a hydrogen atom, or a linear or branched C₁-C₆ alkyl group optionally substituted; a linear or branched C₂-C₆ alkenyl group optionally substituted; a C₃-C₆ cycloalkyl group optionally substituted; a hydroxyl group; an aryl group optionally substituted; a hetero-aryl group optionally substituted; a heterocyclic group optionally substituted;
R₄ and R₅ can individually form a cycle together with R₂;
X represents a nitrogen or sulfur atom;
Z represents a carbon or sulfur atom;
m represents a number ranging from 1 to 5;
n and p represent a number ranging from 0 to 3;
q has the value of 0 for X=sulfur or the value of 1 for X=nitrogen;
s has the value of 1 for Z=carbon or the value of 2 for Z=sulfur;
   for the control of phytopathogen fungi and bacteria and/or the mitigation of abiotic and biotic stress.

Furthermore, an object of the present invention relates to the use of amphoteric compounds having a zwitterionic structure of the betainic type having general formula (I) for the stimulation of the natural defense systems of plants from abiotic and biotic stress and the induction of resistance in the plant itself.

In particular, the use of the compounds having general formula (I) for the control of phytopathogen fungi is curative and/or preventive.

Furthermore, said use for the control of phytopathogen is also effected in genetically modified vegetable varieties.

A further object of the present invention also relates to the use of said compounds having general formula (I) for the control of fungal diseases also on non-living substrates, such as for example, plastic materials, metals, textile fibres, glass, wood, paper, foams, bricks, etc. Said compounds can be applied to the surface of the substrate by means of methods well known in the art, such as for example, spraying, painting, immersion, impregnation, etc., at application doses depending on the kind of material and conditions to which the substrate is subjected.

A C₁-C₂₆ alkyl group refers to a linear or branched C₁-C₂₆ alkyl group, optionally substituted by one or more substituents the same or different.

Examples of this group are: methyl, ethyl, propyl, isopropyl, butyl, isobutyl, tert-butyl, capryl, lauryl, stearyl, eicosyl, hexacosyl.

A C₁-C₂₆ haloalkyl group refers to a linear or branched alkyl group, substituted by one or more halogen atoms, the same or different.

Examples of this group are: fluoromethyl, difluoromethyl, trifluoromethyl, trichloromethyl, 2,2,2-trifluoroethyl, 2,2,2-trichloroethyl, 2,2,3,3-tetrafluoropropyl, 2,2,3,3,3-pentafluoropropyl, perfluoro-octanyl, perfluorododecyl.

A C₁-C₂₆ alkoxyl group refers to a C₁-C₂₆ alkoxyl group, wherein the aliphatic portion is a C₁-C₂₆ alkyl, as previously defined.

Examples of this group are: methoxyl, ethoxyl, isopropoxyl, cyclopropylmethoxyl, lauryloxyl.

A C₁-C₂₆ thioalkyl group refers to a C₁-C₂₆ thioalkyl group, wherein the aliphatic portion is a C₁-C₂₆ alkyl, as previously defined.

Examples of this group are: thiomethyl, thioethyl, thiolauryl, thiocapryl.

A C₂-C₂₆ alkenyl group refers to a linear or branched C₂-C₂₆ alkenyl group, optionally substituted by one or more substituents the same or different.

Examples of this group are: ethenyl, propenyl, butenyl, 1-decenyl, 8-heptadecenyl, 8,11,14-heptadecatrienyl, 8,11-heptadecadienyl.

A C₂-C₂₆ alkinyl group refers to a linear or branched C₂-C₂₆ alkinyl group, optionally substituted by one or more substituents the same or different.

Examples of this group are: ethinyl, propargyl, 1-dodecinyl, 1-octadecinyl.

A C₃-C₃₀ cyclo-alkyl group optionally condensed refers to a cyclo-alkyl group whose ring consists of 3-30 carbon atoms, optionally substituted by one or more substituents the same or different.

Examples of this group are: cyclopropyl, 2,2-dichlorocyclopropyl, cyclobutyl, cyclopentyl, cyclohexyl, decaline, abietyl.

A condensed C₁₇ cyclo-alkyl group of the steroid type refers to a steroid group consisting of 17 carbon atoms, optionally substituted by one or more substituents the same or different.

Examples of this group are: cholanyl, or chenodeoxycholanyl, or ursodeoxycholanyl, or deoxycholanyl, or iodeoxycholanyl, or lithocholanyl.

A C₃-C₃₀ cyclo-alkoxyl group refers to a C₃-C₃₀ cyclo-alkoxyl group wherein the aliphatic portion is a C₃-C₃₀ cyclo-alkyl group as previously defined.

Examples of this group are: cyclopentoxy, cyclohexyloxy, cholesteryl.

A C₁-C₂₆ alkylamine or a C₂-C₂₆ dialkylamine group refers to an alkylamine or dialkylamine group wherein the aliphatic portion is respectively a C₁-C₂₆ or two C₁-C₁₃ alkyl groups as previously defined.

Examples of this group are: methylamine, dimethylamine, ethylamine, isopropylamine, dibutylamine, dioctylamine, hexadecylamine, dodecylamine.

An aryl group refers to an carbocyclic aromatic group optionally substituted by one or more groups the same or different.

Examples of this group are: phenyl, naphthyl, phenanthryl.

A hetero-aryl group refers to a penta- or hexaatomic heterocyclic aromatic group also benzocondensed or heterobicyclic, containing from 1 to 4 hetero-atoms selected from nitrogen, oxygen, sulfur, optionally substituted by one or more groups the same or different.

Examples of hetero-aryl groups are: pyridine, pyrimidine, pyridazine, pyrazine, triazine, tetrazine, quinoline, quinoxaline, quinazoline, furan, thiophene, pyrol, oxazole, thiazole, isoxazole, isothiazole, oxadiazole, thiadiazole, pyrazole, imidazole, triazole, tetrazole, indole, benzofuran, benzothiophene, benzoxazole, benzothiazole, benzoxadiazole, benzothiadiazole, benzopyrazole, benzimidazole, benzotriazole, triazolepyridine, triazolepyrimidine, thiazoltrizole, cumarin.

A heterocyclic group refers to a saturated or unsaturated ring with three to twelve terms, containing at least a heteroatom selected from nitrogen, oxygen, sulfur, optionally condensed with another aromatic or non-aromatic ring.

Examples of heterocyclic rings are: pyrrolidine, piperidine, dihydropyridine, piperazine, 2,6-diketopiperazine, 2-ketoazetidine, morpholine, thiazine, indoline.

A linear or cyclic C₆-C₁₂ group of the saccharide type refers to a carbohydrate group in open or closed form.

Examples of this group are: gluconyl, glucopyranosyl, β-D-fructofuranosyl-α-D-glucopyranosyl, 4-O-β-D-galactopyranosyl-D-glucosyl.

Optionally substituted means, in all parts of the patent, one or more substituents, the same or different, selected from the following groups: halogen atoms; C₁-C₆ alkyls, C₁-C₆ alkoxyls and C₁-C₆ alkylthio, in turn optionally substituted by halogen atoms; C₁-C₆ alkylcarbonyls and C₁-C₆ alkoxycarbonyls, optionally halogenated; aminocarbonyls, C₁-C₆ alkylaminocarbonyls, C₂-C₁₂ dialkylaminocarbonyls, optionally halogenated; carboxyl; C₁-C₆ alkylcarbonyloxy optionally halogenated; cyano; nitro; formyl; hydroxyl; amino; aryl and hetero-aryl optionally substituted.

Examples of compounds having general formula (I) which are interesting for their activity are:
laurylbetaine;
stearylbetaine;
capryl/capric amidopropylbetaine;
cetylbetaine;
laurylhydroxysultaine;
lauryl/cetyl betaine;
lauryl amidopropylbetaine;
cocamidopropylbetaine;
cocamidopropylhydroxysultaine;
cholesterylcarbonylamidopropylbetaine;
cholanylamidopropylbetaine;
chenodeoxycholanylamidopropylbetaine;
deoxycholanylamidopropylbetaine;
*lithocholanylamidopropylbetaine;
cyclohexyloxycarbonylamidopropylbetaine;
gluconylamidopropylbetaine;
N,N-dilaurylaminopropylbetaine;
N-hexadecylureidopropylbetaine;
cocamidopropylmethylacetothetine;
laurylamidopropylmethylacetothetine;
cetylmethylacetothetine;
N,N-dioctylureidopropylbetaine;
laurylamidoethylbetaine;
laurylamidopropyl[L]valinebetaine;
laurylamidopropyl[L]prolinebetaine;
laurylamidopropyl[L]alaninebetaine;
laurylamidopropyl[L]phenylglycinebetaine;
laurylamidopropyl-β-phenylalaninebetaine;
laurylamidopropyl-β-4-chlorophenylalaninebetaine;
laurylamidopropyl-β-alaninebetaine;
cocamidopropyl[L]valinebetaine;
cocamidopropyl[L]prolinebetaine;
cocamidopropyl[L]alaninebetaine;
cocamidopropyl[L]phenylglycinebetaine;
cocamidopropyl-β-phenylalaninebetaine;
cocamidopropyl-β-4-chlorophenylalaninebetaine;
cocamidopropyl-β-alaninebetaine;
decahydro-2-naphthoxycarbonylamidopropylbetaine;
3,5-diterbutylphenylamidopropylbetaine;
3,5-diterbutylphenoxycarbonylamidopropylbetaine;
α-D-glucopyranosyl-β-D-fructofuranosyloxycarbonylamidopropylbetaine;
carnitine.

The compounds having formula (I), when R₁ has the meanings defined above with the exclusion of a C₁-C₂₆ alkoxyl group, or a C₁-C₂₆ alkylthio group, or a C₃-C₃₀ cyclo-alkoxyl group, or a C₁-C₂₆ alkylamine group, or a C₂-C₂₆ dialkylamine group, can be easily obtained according to reaction scheme A for n different from 0 and according to reaction scheme B for n=0:

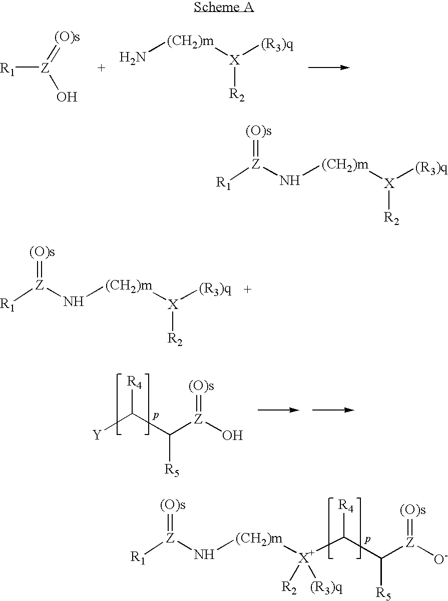

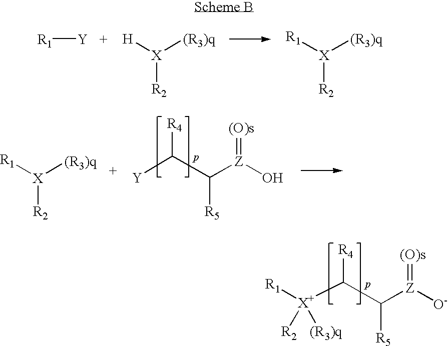

wherein R₁, R₂, R₃, R₄, R₅, X, Z, m, p, q and s have the meanings defined above, Y represents an outgoing group such as a chlorine atom, a bromine atom, an RSO₃— group wherein R represents a C₁-C₆ alkyl or a C₁-C₆ haloalkyl or a phenyl optionally substituted.

The compounds having general formula (I), according to reaction scheme A, for X=nitrogen, can be obtained by condensation of the suitable N′,N′-dialkylamino-N-alkylamine or, for X=sulfur, by condensation of the suitable ω-alkylthioalkylamine with carboxylic acid of a suitable R₁ residue, and a condensing agent, optionally in the presence of a base in an organic or aqueous solvent, according to methods well known in the art, for example in Comprehensive Organic Transformations 1989, R. C. Larock, so as to form the corresponding amide.

The intermediate thus obtained is subsequently subjected to alkylation by reaction with the salt of an alkaline metal, such as for example sodium or potassium, of a suitable organic acid having an outgoing group Y, in water or in an organic solvent, at temperatures ranging from room temperature to 100° C., maintaining the pH at values of around 7.5, by the controlled addition of a solution of a strong base.

The compounds having general formula (I), according to reaction scheme B, for X=nitrogen, can be obtained by alkylation of the suitable N′,N′-dialkylamino-N-alkylamine or, for X=sulfur, by alkylation of the suitable ω-alkylthioalkylamine with the desired R₁ residue having the outgoing group Y, in the presence of a base in an organic or aqueous solvent, according to methods well known in the art, for example in Comprehensive Organic Transformations 1989, R. C. Larock, so as to form the corresponding tertiary amine.

The intermediate thus obtained is subsequently subjected again to alkylation by reaction with the salt of an alkaline metal, such as for example sodium or potassium, of a suitable organic acid having an outgoing group Y, in water or in an organic solvent, at temperatures ranging from room temperature to 100° C., maintaining the pH at values of around 7.5, by the controlled addition of a solution of a strong base.

The compounds having formula (I), when R₁ has the meanings of a C₁-C₂₆ alkoxyl group, or a C₁-C₂₆ alkylthio group, or a C₃-C₃₀ cyclo-alkoxyl group, or a C₁-C₂₆ alkylamine group, or a C₂-C₂₆ dialkylamine group, can be easily obtained according to reaction scheme C for n different from 0:

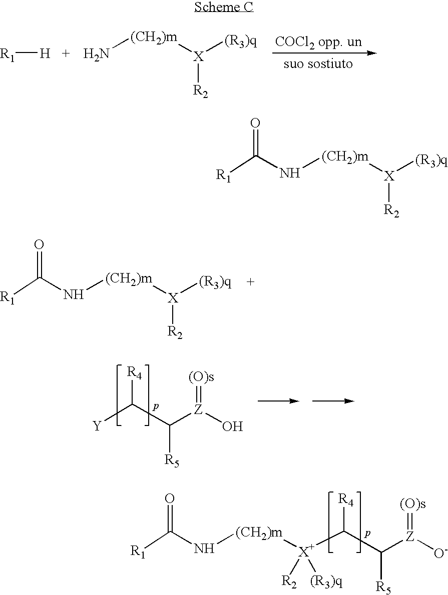

wherein R₁, R₂, R₃, R₄, R₅, X, Z, m, p, q and s have the meanings defined above, Y represents an outgoing group such as a chlorine atom, a bromine atom, an RSO₃⁻ group wherein R represents a C₁-C₆ alkyl or a C₁-C₆ haloalkyl or a phenyl optionally substituted.

The compounds having general formula (I), according to reaction scheme C, for X=nitrogen, can be obtained by reaction of the suitable N′,N′-dialkylamino-N-alkylamine or, for X=sulfur, by reaction of the suitable ω-alkylthioalkylamine with the desired R₁ residue having an alcoholic, or thio-alcoholic, or aminic function when R₁ has the meanings of a C₁-C₂₆ alkoxyl group, or a C₃-C₃₀ cyclo-alkoxyl group, or a C₁-C₂₆ alkylthio group, or a C₁-C₂₆ alkylamine group, or a C₂-C₂₆ dialkylamine group respectively, in the presence of phosgene or one of its functional substitutes, such as, for example, diphosgene, triphosgene, 1,1′-carbonyldiimidazole, in an organic or aqueous solvent, according to methods well known in the art, for example in Comprehensive Organic Transformations 1989, R. C. Larock, so as to form the corresponding carbamate, thiocarbamate or urea.

The intermediate thus obtained is subsequently subjected again to alkylation by reaction with the salt of an alkaline metal, such as for example sodium or potassium, of a suitable organic acid having an outgoing group Y, in water or in an organic solvent, at temperatures ranging from room temperature to 100° C., maintaining the pH at values of around 7.5, by the controlled addition of a solution of a strong base.

The reactions can be conveniently carried out in an aqueous or inert organic solvent, at a temperature ranging from room temperature to the boiling point of the reaction mixture, optionally in the presence of an inorganic or organic base.

Examples of preferred solvents for effecting the reaction are ethers (ethyl ether, isopropyl ether, tetrahydrofuran, dioxane, dimethoxyethane, etc.); esters (ethyl acetate, etc.); chlorinated hydrocarbons (methylene chloride, dichloroethane, chloroform, carbon tetrachloride, etc.); aromatic hydrocarbons (benzene, toluene, xylene, etc.); aliphatic hydrocarbons (hexane, heptane, cyclohexane, etc.); aprotic dipolar solvents (N,N-dimethylformamide, dimethylsulfoxide, sulfolane, etc.).

Examples of preferred inorganic bases are: hydroxides, carbonates of alkaline or alkaline earth metals (sodium, potassium, calcium, etc.).

Examples of preferred organic bases are: pyridine, dimethylaminopyridine, aliphatic amines (triethylamine, etc. cyclic amines (morpholine, piperidine, etc.).

If the substituents R₁, R₂, R₃, R₄, R₅ contain optic or geometric isomerism centres, the compounds having general formula (I) can be present in all possible configurational isomeric forms.

The scope of the present invention therefore also comprises the use of the compounds having general formula (I) as isomeric mixtures in any proportion, and also the formation and use of the single isomers for the control of phytopathogen fungi in the agronomical field.

When deriving from natural extracts, the compounds having general formula (I) can also be present in mixtures of their homologous products and the scope of the present invention consequently also includes the use of the compounds having general formula (I) as mixtures of their homologous products in any proportion, for the control of phytopathogen fungi and bacteria in the agronomical field.

The compounds having general formula (I) can also be present in a hydrated form by the coordination of any number of water molecules, or obtained in aqueous solution and used directly for agronomical purposes.

The compounds having general formula (I) can also contain and possibly coordinate within their structure other metallic cations, such as for example sodium, calcium, potassium, whose number can vary in relation to the preparation method used for the synthesis of the compound having general formula (I).

The scope of the present invention therefore also comprises the use of said solutions of compounds having formula (I), containing said salts for the control of phytopathogen fungi and bacteria in the agronomical field.

The compounds having general formula (I) are capable of controlling numerous fungal and bacterial phytopathogens, also with a reduced sensitivity towards other fungicides.

Examples of phytopathogen fungi and bacteria which can be effectively fought with the compounds having general formula (I) are:
Helminthosporium spp on cereals;
Erysiphe spp on cereals;
Puccinia spp. on cereals;
Plasmopara viticola on vines;
Pythium spp on vegetables;
Phytophthora spp. on vegetables;
Rhynchosporium on cereals;
Septoria spp. on cereals;
Sphaerotheca fuliginea on cucurbits (for example cucumbers);
Podosphaera leucotricha on apple trees;
Pyricularia oryzae on rice;
Uncinula necator on vines;
Venturia spp. on fruit trees;
Botrytis cinerea on vines and vegetables;
Fusarium spp. on cereals;
Alternaria spp. on fruit trees and vegetables;
Cercospora spp. on sugar beet;
Xantomonas;
Bacillus spp.

The compounds having general formula (I) are capable of exerting a fungicidal action of both a curative and preventive nature and have a low or zero phytotoxicity.

A further object of the present invention therefore relates to a method for controlling phytopathogen fungi and bacteria in agricultural crops by the application of the amphoteric compounds with a zwitterionic structure of the betainic type having general formula (I) having a direct fungicidal and bacterial activity and a method for the stimulation of the natural defense systems of plants from abiotic stress (temperature, salinity, drought, etc.) and biotic stress and the induction of resistance in the plant itself by the application of the amphoteric compounds with a zwitterionic structure of the betainic type having general formula (I).

The quantity of compound to be applied for obtaining the desired effect can vary in relation to various factors such as, for example, the compound used, the crop to be preserved, the type of pathogen, the degree of infection, the climatic conditions, the application method and the formulation adopted.

Doses of compound ranging from 10 g to 5 kg per hectare generally provide a sufficient control.

For practical uses in agriculture, it is often useful to adopt fungicidal compositions containing one or more amphoteric compounds having a zwitterionic structure of the betainic type having general formula (I).

The application of these compositions can be effected on all parts of the plant, for example on the leaves, stems, branches and roots, or on the seeds themselves before sowing, or on the ground in which the plant grows.

Compositions can be used in the form of dry powders, wettable powders, emulsifying concentrates, microemulsions, pastes, granulates, solutions, suspensions, etc.: the choice of the type of composition will depend on the specific use.

The compositions are prepared in the known way, for example by diluting or dissolving the active substance with a solvent medium and/or a solid diluent, possibly in the presence of surface-active agents.

Solid diluents or supports which can be used are, for example: silica, kaolin, bentonite, talc, infusorial earth, dolomite, calcium carbonate, magnesia, gypsum, clays, synthetic silicates, attapulgite, sepiolite.

Liquid diluents which can be used, in addition to water, are, for example, aromatic organic solvents (xylols or alkyl benzene mixtures, chlorobenzene, etc.), paraffins (oil fractions), alcohols (methanol, propanol, butanol, octanol, glycerin, etc.), esters (ethyl acetate, isobutyl acetate, etc.), ketones (cyclohexanone, acetone, acetophenone, isophorone, ethylamylketone, etc.), amides (N,N-dimethylformamide, N-methylpyrrolidone, etc.).

Surface-active agents which can be used are salts of sodium, calcium, triethylamine or triethanolamine, alkylsulfonates, alkylaryl-sulfonates, polyethoxylated alkylphenols, polyethoxylated esters of sorbitol, ligninsulfonates, etc.

The compositions can also contain special additives for particular purposes, for example adhesion agents such as gum arabic, polyvinyl alcohol, polyvinylpyrrolidone, polyacrylates, etc.

It has also be found, in agronomical practice, that the fungicidal action of compounds having general formula (I) is particularly effective when combined with that of numerous other fungicidal active principles thus creating an excellent instrument for anti-resistance strategies, allowing the applicative doses to be further lowered and stimulating the natural defense of plants.

More specifically, a high synergy has been observed by mixing the compounds having general formula (I) with other compounds also known to be capable of stimulating the natural defense of plants such as salicylic acid, acetylsalicylic acid, copper (II) salt of acetylsalicylic acid ASA₂Cu, 2,6-dichloroisonicotinic acid (INA), 1′Smethylester of benzo[1,2,3]thiadiazolyl-7-thiocarboxylic acid (BTH), saccharine, thus enhancing and modulating the biological activity in an effective and safe manner.

In particular, an increased biological activity of the following compounds has been observed:
phosphorous acid, its salts and mixtures thereof, such as for example, K₂HPO₃, KH₂PO₃, Na₂HPO₃, NaH₂PO₃, (NH₄)₂HPO₃, NH₄H₂PO₃;
benalaxyl (in its racemic form or as an optically active R isomer);
fungicidal dipeptide IR5885 (in its racemic form or as an optically active R isomer);
tetraconazole (in its racemic form or as an optically active R isomer);
resistance inducers such as for example: salicylic acid, its derivatives and cupric salts, acetylsalicylic acid, its derivatives and cupric salts, such as for example, the copper (II) salt of acetylsalicylic acid ASA₂Cu, the copper (II) salt of salicylic acid SA₂Cu, the copper (II) salt of salicylic acid SACu, 2,6-dichloroisonicotinic acid (INA), 1′S-methylester of benzo[1,2,3]thiadiazolyl-7-thiocarboxylic acid (BTH), saccharine;
cupric salts such as for example: copper hydroxide, copper oxychloride, cuprocalcium oxychloride, tribasic copper sulfate;
iprovalicarb;
benthiavalicarb-isopropyl;
cyazofamide;
   when mixed with the compounds having general formula (I).

Said fungicidal compounds are commercial compounds or almost ready to be commercialized.

A description thereof can be easily found in technical literature, for example in “The Pesticide Manual”, 2000, XII edition, British Crop Council Ed., in www. Agrowreports. Com.

IR5885, dipeptide with a fungicidal activity refers to one of the compounds among those claimed in patent application EP 1028125.

An object of the present invention therefore relates to the use of said compositions comprising at least one amphoteric compound having general formula (I) with one or more of the following fungicidal compounds:
phosphorous acid, its salts and mixtures thereof, such as for example, K₂HPO₃, KH₂PO₃, Na₂HPO₃, NaH₂PO₃, (NH₄)₂HPO₃, NH₄H₂PO₃;
benalaxyl (in its racemic form or as an optically active R isomer);
the fungicidal dipeptide IR5885 (in its racemic form or as an optically active R isomer);
tetraconazole (in its racemic form or as an optically active R isomer);
resistance inducers such as for example: salicylic acid, its derivatives and cupric salts, acetylsalicylic acid, its derivatives and cupric salts, such as for example, the copper (II) salt of acetylsalicylic acid ASA₂Cu, the copper (II) salt of salicylic acid SA₂Cu, the copper (II) salt of salicylic acid SACu, 2,6-dichloroisonicotinic acid (INA), 1′S-methylester of benzo[1,2,3]thiadiazolyl-7-thiocarboxylic acid (BTH), saccharine;
cupric salts such as for example: copper hydroxide, copper oxychloride, cuprocalcium oxychloride, tribasic copper sulfate;
iprovalicarb;
benthiavalicarb-isopropyl;
cyazofamide;
   which have a surprising higher biological activity than that envisaged by simple mixing of the two active principles.

Preferred compositions according to the present invention are selected from:
glycinebetaine and K₂HPO₃
glycinebetaine and KH₂PO₃,
cocamidopropyl betaine and K₂HPO₃
cocamidopropyl betaine and KH₂PO₃,
cocamidopropyl betaine and tetraconazole;
cocamidopropyl betaine and tetraconazole R isomer;
cocamidopropyl betaine and IR5885;
cocamidopropyl betaine and iprovalicarb;
cocamidopropyl betaine and benthiavalicarbisopropyl;
cocamidopropyl betaine and cyazofamide;
cocamidopropyl betaine and R isomer IR5885;
cocamidopropyl betaine, IR5885 and K₂HPO₃— KH₂PO₃;
cocamidopropyl betaine, R isomer IR5885 and K₂HPO₃—KH₂PO₃;
glycine betaine, K₂HPO₃— KH₂PO₃ and IR5885;
glycine betaine, K₂HPO₃— KH₂PO₃ and iprovalicarb;
glycine betaine, K₂HPO₃— KH₂PO₃ and benthiavalicarbisopropyl;
glycine betaine, K₂HPO₃— KH₂PO₃ and cyazofamide;
glycine betaine, K₂HPO₃— KH₂PO₃ and R isomer IR5885;
cocamidopropyl betaine and ASA₂CU;
cocamidopropyl betaine and SA₂CU;
cocamidopropyl betaine and SACu,
carnitine and K₂HPO₃,
carnitine and KH₂PO₃;
carnitine and K₂HPO₃— KH₂PO₃ and IR5885.

The concentration of active principles in the above compositions can vary within a wide range depending on the active compounds, the applications for which they are destined, the environmental conditions and the type of formulation adopted.

The concentration of active principle generally ranges from 1% to 90%, preferably from 5 to 50%.

The following examples are provided for a better understanding of the invention for illustrative and non-limiting purposes of the present invention.

### EXAMPLE 1

### Preparation of Laurylamidopropyl-N,N-Dimethylamine

4.67 g of 3-dimethylamino-1-propylamine are added to a solution of 10 g of lauroylchloride in 50 ml of methylene chloride and 4.74 ml of triethylamine. The mixture is kept under stirring at room temperature for a night. The product obtained is extracted, washed with water, anhydrified with Na₂SO₄ obtaining, after drying, 12 g of the desired compound (yield: 93%).

Elemental analysis [% found (theoretical)]=C, 71.2 (71.6); H, 12.5 (12.6); N, 9.5 (9.8).

### EXAMPLE 2

### Preparation of Eicosyldimethylamine

??? of 3-dimethylamino-1-propylamine are added to a solution of 10 g of eicosylbromide in water and 10.5 ml of dimethylamine at 40% in an aqueous solution. The mixture is kept under stirring at room temperature for a night. The product obtained is extracted, washed with water, anhydrified with Na₂SO₄ obtaining, after drying, 8.1 g of the desired compound (yield: 90%).

Elemental analysis [% found (theoretical)]=C, 80.9 (81.1); H, 14.3 (14.7); N, 4.5 (4.3).

### EXAMPLE 3

### Preparation of Cholesterylamidopropyldimethylamine

3.41 g of 3-dimethylamino-1-propylamine are added to a solution of 15 g of cholesterylchloroformiate in 70 ml of methylene chloride and 3.49 ml of triethylamine. The mixture is kept under stirring at room temperature for a night. The product obtained is extracted, washed with water, anhydrified with Na₂SO₄ obtaining, after drying, 15.8 g of the desired compound (yield: 92%).

Elemental analysis [% found (theoretical)]=C, 77.0 (76.8); H, 11.9 (11.2); N, 5.1 (5.4).

### EXAMPLE 4

### Preparation of Laurylamidopropyl Betaine (Compound 7)

12 g of laurylamidopropyl-N,N-dimethylamine in 32 ml of water are charged into a reactor and 4.9 g of sodium monochloroacetate are added. The reaction mixture is slowly heated to 98° C. and the pH is maintained at around 7.5 by the continuous addition of a 50% by weight solution of sodium hydroxide. After about 5 hours the starting products are completely used up and the solution obtained is used as such.

Analogously to what is described in the examples, the following compounds were prepared:

TABLE 1

| Number | Compound |
|---|---|
| 1 | Laurylbetaine |
| 2 | Stearylbetaine |
| 3 | capryl/capric amidopropylbetaine |
| 4 | Cetylbetaine |
| 5 | Laurylhydroxysultaine |
| 6 | lauryl/cetyl betaine |
| 7 | Laurylamidopropylbetaine |
| 8 | Cocamidopropylbetaine |
| 9 | Cocamidopropylhydroxysultaine |
| 10 | Cholesterylcarbonylamidopropylbetaine |
| 11 | Cholanylamidopropylbetaine |
| 12 | Chenodeoxycholanylamidopropylbetaine |
| 13 | Deoxycholanylamidopropylbetaine |
| 14 | Lithocholanylamidopropylbetaine |
| 15 | cyclohexyloxycarbonylamidopropylbetaine |
| 16 | Gluconylamidopropylbetaine |
| 17 | N,N-dilaurylaminopropylbetaine |
| 18 | N-hexadecylureidopropylbetaine |
| 19 | Cocamidopropylmethylacetothetine |
| 20 | Laurylamidopropylmethylacetothetine |
| 21 | Cetylmethylacetothetine |
| 22 | N,N-dioctylureidopropylbetaine |
| 23 | Laurylamidoethylbetaine |
| 24 | laurylamidopropyl[L]valinebetaine |
| 25 | laurylamidopropyl[L]prolinebetaine |
| 26 | laurylamidopropyl[L]alaninebetaine |
| 27 | laurylamidopropyl[L]phenylglycinebetaine |
| 28 | laurylamidopropyl- β-phenylalaninebetaine |
| 29 | laurylamidopropyl- β-4-chlorophenylalaninebetaine |
| 30 | laurylamidopropyl- β-alaninebetaine |
| 31 | Cocamidopropyl[L]valinebetaine |
| 32 | Cocamidopropyl[L]prolinebetaine |
| 33 | cocamidopropyl[L]alaninebetaine |
| 34 | Cocamidopropyl[L]phenylglycinebetaine |
| 35 | Cocamidopropyl-β-phenylalaninebetaine |
| 36 | Cocamidopropyl- β-4-chlorophenylalaninebetaine |
| 37 | Cocamidopropyl- β-alaninebetaine |
| 38 | decahydro-2-naphthoxycarbonylamidpropylbetaine |
| 39 | 3,5-diterbutylphenylamidopropylbetaine |
| 40 | 3,5-diterbutylphenoxycarbonylamidopropylbetaine |
| 41 | α-D-glucopyranosyl-β-D- |
| | fructofuranosyloxycarbonylamidopropylbetaine |

### EXAMPLE 5

### Determination of the Fungicidal Activity Against Peronospora of Vines (Plasmopara viticola)

Vine leaves (cultivar Dolcetto), grown in vases in a conditioned environment (20±1° C., 70% relative humidity) are treated by spraying both sides of the leaves with compounds 1, 2 and 3, dispersed in a hydroacetone solution at 20% by volume in acetone.

After remaining 24 hours in a conditioned environment, the plants were sprayed on both sides of the leaves with an aqueous suspension of conidia of Plasmopara viticola (20,000 conidia per cm³).

The plants are kept in a humidity saturated environment at 21° C. for the incubation period of the fungus.

At the end of this period (7 days), the fungicidal activity is evaluated according to an evaluation percentage scale from 0 (completely infected plant) to 100 (healthy plant).

TABLE 2
7-day preventive activity on Plasmopara viticola
of the compounds having general formula (I)

| Compound Nr. | Activity 250 ppm | Activity 125 ppm |
|---|---|---|
| 5 | 99 | 85 |
| 7 | 100 | 95 |
| 8 | 100 | 98 |
| 9 | 100 | 95 |
| 10 | 95 | 90 |
| 11 | 94 | 90 |
| 24 | 99 | 88 |
| 25 | 100 | 95 |
| 26 | 95 | 90 |
| 28 | 99 | 90 |
| 29 | 95 | 90 |
| 30 | 90 | 85 |
| 31 | 98 | 85 |
| 32 | 95 | 88 |
| 33 | 96 | 85 |
| 34 | 97 | 88 |
| 35 | 95 | 89 |
| 36 | 94 | 85 |
| 37 | 98 | 86 |

TABLE 3
7-day preventive activity on Plasmopara viticola of mixtures of
the compounds having general formula (I) with other fungicides.

| | Activity | | Activity | | Activity | |
|---|---|---|---|---|---|---|
| Mixture | (dose ppm) | | (dose ppm) | | (dose ppm) | |
| Glycine betaine | 30 | (30 ppm) | | | | |
| K₂HPO₃—KH₂PO₃ | | | 40 | (500 ppm)* | | |
| Glycine betaine + | | | | | 90 | (30 + 500 ppm) |
| K₂HPO₃—KH₂PO₃ | | | | | | |
| Tetraconazole | 20 | (30 ppm) | | | | |
| Compound Nr. 8 | | | 30 | (30 ppm) | | |
| Tetracon. + Comp. Nr. 8 | | | | | 95 | (30 + 30 ppm) |
| IR5885 | 41 | (0.45 ppm) | | | | |
| IR5885 + Comp. Nr. 8 | | | | | 100 | (0.45 + 30 ppm) |
| IR5885 | 15 | (0.22 ppm) | | | | |
| IR5885 + Comp. Nr. 8 + | | | | | 100 | (0.22 + 30 + 500 ppm) |
| K₂HPO₃—KH₂PO₃ | | | | | | |

*when the dose in ppm relates to potassium phosphite, this is expressed in equivalent phosphorous acid.

### EXAMPLE 6

### Determination of the Fungicidal Activity Against Oidium of Wheat (Erysiphe graminis)

Leaves of wheat plants (cultivar Gemini), grown in vases in a conditioned environment (20±1° C., 70% relative humidity) are treated by spraying both sides of the leaves with compounds 1, 2 and 3, dispersed in a hydroacetone solution at 20% by volume in acetone.

After remaining 24 hours in a conditioned environment, the plants were sprayed on both sides of the leaves with an aqueous suspension of conidia of Erysiphe graminis (200,000 conidia per cm³).

The plants are kept in a humidity saturated environment at a temperature ranging from 18 to 24° C. for the incubation period of the fungus.

At the end of this period (12 days), the fungicidal activity is evaluated according to an evaluation percentage scale from 0 (completely infected plant) to 100 (healthy plant).

TABLE 4
5-day preventive activity on Erysiphe graminis of mixtures of the
compounds having general formula (I) with other fungicides.

| | Activity | | Activity | Activity | |
|---|---|---|---|---|---|
| Mixture | (dose ppm) | | (dose ppm) | (dose ppm) | |
| Tetraconazole | 48 | (1.8 ppm) | | | |
| Compound | | | 20 (500 ppm) | | |
| Nr. 8 | | | | | |
| Tetracon. + | | | | 97 | (1.8 + 500 ppm) |
| Comp. Nr. 8 | | | | | |
| IR5885 | 20 | (500 ppm) | | | |
| IR5885 + | | | | 93 | (500 + 500 ppm) |
| Comp. Nr. 8 | | | | | |

### EXAMPLE 7

### Determination of the Fungicidal Activity Against Wheat Rust (Puccinia recondita)

Leaves of wheat plants (cultivar Gemini), grown in vases in a conditioned environment (20±1° C., 70% relative humidity) are treated by spraying both sides of the leaves with compounds 1, 2 and 3, dispersed in a hydroacetone solution at 20% by volume in acetone.

After remaining 24 hours in a conditioned environment, the plants were sprayed on both sides of the leaves with an aqueous suspension of conidia of Puccinia recondita (200,000 conidia per cm³).

The plants are kept in a humidity saturated environment at a temperature ranging from 18 to 24° C. for the incubation period of the fungus.

At the end of this period (14 days), the fungicidal activity is evaluated according to an evaluation percentage scale from 0 (completely infected plant) to 100 (healthy plant).

TABLE 5
5-day preventive activity on Puccinia recondita of mixtures of
the compounds having general formula (I) with other fungicides.

| | Activity | | Activity | Activity | |
|---|---|---|---|---|---|
| Mixture | (dose ppm) | | (dose ppm) | (dose ppm) | |
| Tetraconazole | 58 | (30 ppm) | | | |
| Compound | | | 29 (125 ppm) | | |
| Nr. 8 | | | | | |
| Tetracon. + | | | | 92 | (30 + 125 ppm) |
| Comp. Nr. 8 | | | | | |
| IR5885 | 15 | (500 ppm) | | | |
| IR5885 + | | | | 81 | (500 + 125 ppm) |
| Comp. Nr. 8 | | | | | |

### EXAMPLE 8

### Determination of the Gene Response of the Compounds Having General Formula (I) and their Mixtures with Other Fungicides

Four-week-old seedlings of arabidopsis thaliana were treated with the compounds having general formula (I) or their mixtures with other fungicides and the leaves were collected after 24 hours of treatment.

The total RNA was extracted from 0.05 g of fresh tissue using the “Genelute mammalian total RNA kit (Sigma)” according to the protocol indications. The cDNA were synthesized using “RevertAid™ M-MuLV Reverse Transcriptase” commercialized by Fermentas Life Sciences according to the following protocol: 2 μg of total RNA were mixed with 0.5 μg of oligo(dT) 18.

Deionized water (nuclease free) was then added to bring the reaction volume to 11 μl, the reaction was subsequently incubated at 70° C. for 5 minutes and then cooled in ice.

The following reagents were then added to the mixture:

4 μl of 5× reaction buffer, 10 mM of dNTP mix, 20 units of Ribonuclease inhibitor.

The reaction was incubated at 37° C. for 5 minutes, 200 units of RevertAid™ M-MuLV Reverse Transcriptase were subsequently added to the mixture and the reaction was incubated at 42° C. for 60 minutes.

The reaction was then blocked by inactivation of the enzyme at 70° C. for 10 minutes.

### PCR Analysis

A quantitative PCR analysis was effected on the cDNA using a mixture of primer/competimers of the ribosomal RNA 18S as internal standard in a ratio of 9:1.

The sequences of the primers used for the PCR reaction are listed below:

| | |
|---|---|
| PR1 fw: | 5′ GTAGCTCTTGTAGGTGCTCT 3′ |
| PR1 rev: | 5′ CATCCTGCATATGATGCTCC 3′ |

The PCR reactions were carried out in 25 μl with the following components:
cDNA: 0.5 μl
10× Reaction buffer: 2.5 μl
50 mM MgCl₂=0.75 μl
2.5 mM dNTPs: 0.5 μl
5 μM 18S Primer:Competimer mix (9:1 ratio): 0.5 μl
12.5 μM Gene specific primer forward: 0.5 μl
12.5 μM Gene specific primer reverse: 0.5 μl
Euroclone Taq (5 u/μl): 0.25 μl)
After 2 minutes of denaturation at 94° C. the following amplification program was effected for 35 cycles:
94° C.: 30 sec
annealing temp PR1:48° C.:30 sec
72° C.: 1 min.

An additional cycle at 72° C. for 10 min. was subsequently effected.

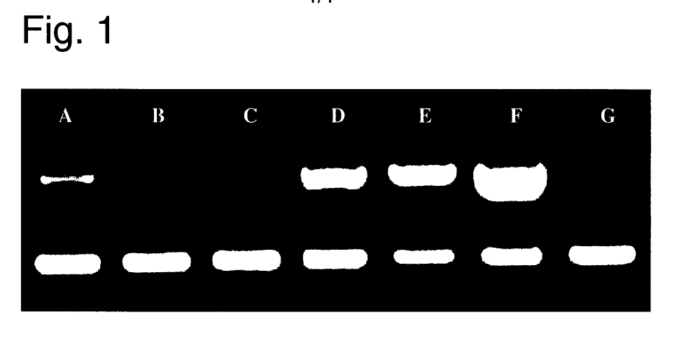

In FIG. 1, wherein
A) ASA₂Cu at 12.5 ppm
B) glycine betaine at 800 ppm
C) glycine betaine at 1600 ppm
D) compound Nr. 8 at 800 ppm
E) compound Nr. 8 at 1600 ppm
F) ASA₂Cu at 12.5 ppm+compound Nr. 8 at 800 ppm
G) blank for control a comparison with the blank and glycine betaine distinctively showed the exceptional gene response of compound Nr. 8 alone and in a mixture with the copper (II) salt of acetylsalicylic acid (ASA₂Cu).

1. An amphoteric compound characterized by a zwitterionic structure of the betaine type having general formula (I), 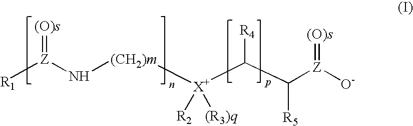
(D wherein:
R₁ represents a linear or branched C₁-C₂₆ alkyl group optionally substituted; a linear or branched C₁-C₂₆ haloalkyl group optionally substituted; a linear or branched C₁-C₂₆ alkoxyl group optionally substituted; a linear or branched C₁-C₂₆ alkylthio group optionally substituted; a linear or branched C₂-C₂₆ alkenyl group optionally substituted; a linear or branched C₂-C₂₆ alkinyl group optionally substituted; a C₃-C₃₀ cycloalkyl group optionally condensed or a condensed C₁₋₇ cycloalkyl group of the steroid type optionally substituted; a C₃-C₃₀ cycloalkoxyl group optionally condensed and optionally substituted; a heterocyclic group optionally substituted; an aryl group optionally substituted; a heteroaryl group optionally substituted; a linear or cyclic C₆-C₁₂ group of the saccharide type optionally substituted; a C₁-C₂₆ alkylamine group or a C₂-C₂₆ dialkylamine optionally substituted for n different from 0; —R₂ and R₃, the same or different, represent a C₁-C₃ alkyl group optionally substituted;
R₄ and R₅, the same or different, represent a hydrogen atom, or a linear or branched C₁-C₆ alkyl group optionally substituted; a linear or branched C₂-C₆ alkenyl group optionally substituted; a C₃-C₆ cycloalkyl group optionally substituted; a hydroxyl group, an aryl group optionally substituted; a heteroaryl group optionally substituted; a heterocyclic group optionally substituted; R₄ and R₅ can individually form a cycle together with R₂;
X represents a nitrogen or sulfur atom;
Z represents a carbon or sulfur atom;
m represents a number ranging from 1 to 5; —n and p represent a number ranging from 0 to 3;
q has the value of 0 for X=sulfur or the value of 1 for X=nitrogen;
s has the value of 1 for Z=carbon or the value of 2 for Z=sulfur.

2. The compound according to claim 1, characterized in that the linear or branched Ci-C₂₆ alkyl group is selected from methyl, ethyl, propyl, isopropyl, butyl, isobutyl, tert-butyl, capryl, lauryl, stearyl, eicosyl, hexacosyl.

3. The compound according to claim 1, characterized in that the C₁-C₂₆ haloalkyl group is selected from fluoromethyl, difluoromethyl, trifluoromethyl, trichloromethyl, 2,2,2-trifluoroethyl, 2,2,2-trichloroethyl, 2,2,3,3-tetrafluoropropyl, 2,2,3,3,3-pentafluoropropyl, perfluorooctanyl, perfluorododecyl.

4. The compound according to claim 1, characterized in that the C₁-C_{2S} alkoxyl group is selected from methoxyl, ethoxyl, isopropoxyl, cyclopropylmethoxyl, lauryloxyl.

5. The compound according to claim 1, characterized in that the C₁-C₂₆ thioalkyl group is selected from thiomethyl, thioethyl, thiolauryl, thiocapryl.

6. The compound according to claim 1, characterized in that the C₂-C₂₆ alkenyl group is selected from ethenyl, propenyl, butenyl, 1-decenyl, 8-heptadecenyl, 8,11,14-heptadecatrienyl, 8,11-heptadecadienyl.

7. The compound according to claim 1, characterized in that the C₂-C₂₆ alkinyl group is selected from ethinyl, propargyl, 1-dodecinyl, 1-octadecinyl.

8. The compound according to claim 1, characterized in that the C₃-C₃₀ cycloalkyl group optionally condensed is selected from cyclopropyl, 2,2-dichlorocyclopropyl, cyclobutyl, cyclopentyl, cyclohexyl, decaline, abietyl.

9. The compound according to claim 1, characterized in that the condensed C₁₋₇ cycloalkyl group of the steroid type is selected from cholanyl, or chenodeoxycholanyl, or ursodeoxycholanyl, or deoxycholanyl, or iodeoxycholanyl, or lithocholanyl.

10. The compound according to claim 1, characterized in that the C₃-C₃₀ cycloalkoxyl group is selected from cyclopentoxy, cyclohexyloxy, cholesteryl.

11. The compound according to claim 1, characterized in that the C₁-C₂₆ alkylamine or a C₂-C₂₆ dialkylamine group are selected from methylamine, dimethylamine, ethylamine, isopropylamine, dibutylamine, dioctylamine, hexadecylamine, didecylamine.

12. The compound according to claim 1, characterized in that the aryl group is selected from phenyl, naphthyl, phenanthryl.

13. The compound according to claim 1, characterized in that the heteroaryl group is selected from pyridine, py rimidine, pyridazine, pyrazine, triazine, tetrazine, quinoline, quinoxaline, quinazoline, furan, thiophene, pyrol, oxazole, thiazole, isoxazole, isothiazole, oxadiazole, thiadiazole, pyrazole, imidazole, triazole, tetrazole, indole, benzofuran, benzothiophene, benzoxazole, benzothiazole, benzoxadiazole, benzothiadiazole, benzopyrazole, benzimidazole, benzotriazole, triazolepyridine, triazolepyrimidine, thiazoltrizole, cumarin.

14. The compound according to claim 1, characterized in that the heterocyclic group is selected from pyrrolidine, piperidine, dihydropyridine, piperazine, 2,6-diketopiperazine, 2-ketoazetidine, morpholine, thiazine, indoline.

15. The compound according to claim 1, characterized in that the linear or cyclic C₆-C₁₂ group of the saccharide type is selected from gluconyl, glucopyranosyl, β-D-fructofuranosyl-α-D-glucopyranosyl, 4-O-β-D-galactopyranosyl-D-glucosyl.

16. The compound according to claim 1, characterized in that it is selected from:
laurylbetaine
stearylbetaine;
capryl/capric amidopropylbetaine;
cetylbetaine;
laurylhydroxysultaine;
lauryl/cetyl betaine;
laurylamidopropylbetaine;
cocamidopropylbetaine;
cocam idopropylhydroxysu Iltaine;
cholesterylcarbonylamidopropylbetaine;
cholanylamidopropylbetaine;
chenodeoxycholanylamidopropylbetaine;
deoxycholanylamidopropylbetaine;
lithocholanylamidopropylbetaine;
cyclohexyloxycarbonylam idopropylbetaine;
gluconylamidopropylbetaine;
N,N-dilaurylaminopropylbetaine;
N-hexadecylureidopropylbetaine;
cocamidopropylmethylacetothetine
laurylamidopropylmethylacetothetine
cetylmethylacetothetine,-
N,N-dioctylureidopropylbetaine;
laurylamidoethylbetaine;
laurylamidopropyl [L] valinebetaine
laurylamidopropyl [L] prolinebetaine;
laurylamidopropyl [L] alaninebetaine;
laurylamidopropyl [L] phenylglycinebetaine;
laurylamidopropyl-β-phenylalaninebetaine,
laurylamidopropyl-β-4-chlorophenylalaninebetaine;
laurylamidopropyl-β-alaninebetaine
cocamidopropyl [L] valinebetaine;
cocamidopropyl [L] prolinebetaine;
cocamidopropyl [L] alaninebetaine;
cocamidopropyl [L] phenylglycinebetaine;
cocamidopropyl-β-phenylalan inebetaine;
cocamidopropyl-O-4-chlorophenylalaninebetaine,
cocamidopropyl-β-alaninebetaine;
decahydro-2-naphthoxycarbonylamidopropylbetaine;
3,5-diterbutylphenylamidopropylbetaine;
3,5-diterbutylphenoxycarbonylamidopropylbetaine;
α-D-glucopyranosyl-1-D-fructofuranosyIoxycarbonylamido-propylbetaine;
carnitine.

17. The compound according to claim 1, characterized in that it has all configurational isomeric forms, when the substituents R₁, R₂, R₃, R₄, R₅ contain optic or geometric isomerism centres.

18. The compound according to claim 1, characterized in that it consists of mixtures of homologous products in any proportion, when the compound derives from natural extracts.

19. The compound according to claim 1, characterized in that it is present in hydrated form by coordination of any number of water molecules.

20. The compound according to claim 1, characterized in that it also contains and possibly coordinates in the structure other metallic cations such as sodium, calcium, potassium, in a variable number depending on the preparation method used for the synthesis of the compound having general formula (I).

21. Use of an amphoteric compound having a zwitterionic structure of the betaine type having general formula (I), 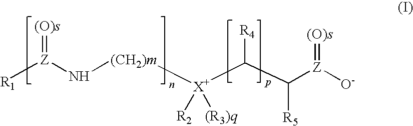
(D wherein:
R₁ represents a linear or branched C₁-C₂₆ alkyl group optionally substituted; a linear or branched C₁-C₂₆ haloalkyl group optionally substituted; a linear or branched C₁-C₂₆ alkoxyl group optionally substituted; a linear or branched C₁-C₂₆ alkylthio group optionally substituted; a linear or branched C₂-C₂₆ alkenyl group optionally substituted; a linear or branched C₂-C₂₆ alkinyl group optionally substituted; a C₃-C₃₀ cycloalkyl group optionally condensed or a condensed C₁₋₇ cycloalkyl group of the steroid type optionally substituted; a C₃-C₃₀ cycloalkoxyl group optionally condensed and optionally substituted;
a heterocyclic group optionally substituted; an aryl group optionally substituted; a heteroaryl group optionally substituted; a linear or cyclic C₆-C₁₂ group of the saccharide type optionally substituted; a C₁-C₂₆ alkylamine group or a C₂-C₂₆ dialkylamine optionally substituted for n different from 0; —R₂ and R₃, the same or different, represent a C₁-C₃ alkyl group optionally substituted; —R₄ and R₅, the same or different, represent a hydrogen atom, or a linear or branched C₁-C₆ alkyl group optionally substituted; a linear or branched C₂-C₆ alkenyl group optionally substituted; a C₃-C₆ cycloalkyl group optionally substituted; a hydroxyl group; an aryl group optionally substituted; a heteroaryl group optionally substituted; a heterocyclic group optionally substituted; R₄ and R₅ can individually form a cycle together with R₂; —X represents a nitrogen or sulfur atom;
Z represents a carbon or sulfur atom;
m represents a number ranging from 1 to 5;
n and p represent a number ranging from 0 to 3
q has the value of 0 for X=sulfur or the value of 1 for X=nitrogen;
s has the value of 1 for Z=carbon or the value of 2 for Z=sulfur; for the control of phytopathogen fungi and bacteria and/or the mitigation of abiotic and biotic stress.

22. (canceled)

23. Use of the compounds according to claim 1, for the stimulation of the natural defense systems of plants from abiotic and biotic stress and the induction of resistance in the plants themselves.

24. Use according to claim 21, characterized in that said use is curative and/or preventive.

25. Use according to claim 21, wherein the compound having general formula (I) is used in a quantity ranging from 10 g to 5 kg per hectare.

26. Use according to claim 21 of compounds having general formula (I) as single isomers or as isomeric mixtures in any proportion.

27. Use according to claim 21 in genetically modified vegetable varieties.

28. Use of a compound according to claim 1 for the control of fungal diseases on non-living substrates, such as plastic materials, metals, textile fibres, glass, wood, paper, foams, bricks.

29. The use according to claim 28, by application of the substrate to the surface by spraying, painting, immersion, impregnation.

30. A method for the control of phytopathogen fungi and bacteria and/or the mitigation of abiotic and biotic stress in agricultural crops by the application of the amphoteric compounds with a zwitterionic structure of the betaine type having general formula (I) according to claim 1.

31. A method for the stimulation of the natural defense systems of plants from abiotic and biotic stress and the induction of resistance in the plants themselves in agricultural crops by the application of the amphoteric compounds with a zwitterionic structure of the betaine type having general formula (I) according to claim 1.

32. A fungicidal composition comprising one or more amphoteric compounds having a zwitterionic structure of the betaine type having general formula (I) according to claim.

33. The composition according to claim 32, characterized in that it contains other active principles.

34. The composition according to claim 33, characterized in that it contains at least one of the following products as further active principles:
phosphorous acid, its derivatives, its salts and mixtures thereof, such as for example, K₂HPO₃, KH₂PO₃, Na₂HPO₃, NaH₂PO₃, (NH₄)₂HPO₃, NH₄H₂PO₃, Fosetyl aluminium;
benalaxyl (in its racemic form or as an optically active R isomer)
fungicidal dipeptide IR5885 (in its racemic form or as an optically active R isomer)
tetraconazole (in its racemic form or as an optically active R isomer)
resistance inducers such as for example: salicylic acid, its derivatives and cupric salts, acetylsalicylic acid, its derivatives and cupric salts, such as for example, the copper (II) salt of acetylsalicylic acid ASA₂Cu, the copper (II) salt of salicylic acid SA₂Cu, the copper (II) salt of salicylic acid SACu, 2,6-dichloroisonicotinic acid (INA), 1′S-methylester of benzo[1, 2, 3] thiadiazolyl-7-thiocarboxylic acid (BTH), saccharine;
cupric salts such as for example: copper hydroxide, copper oxychloride, cuprocalcium oxychloride, tribasic copper sulfate;
iprovalicarb;
benthiavalicarb-isopropyl;
cyazofamide.

35. The composition according to claim 34, characterized in that said composition is selected from: glycinebetaine and K₂HPO₃; glycinebetaine and KH₂PO₃; glycinebetaine and Fosetyl aluminium; cocamidopropylbetaine and K₂HPO₃; cocamidopropylbetaine and KH₂PO₃; cocamidopropylbetaine and Fosetyl aluminium; cocamidopropylbetaine and tetraconazole; —cocamidopropylbetaine and tetraconazole R isomer; cocamidopropylbetaine and IR5885; cocamidopropylbetaine and iprovalicarb; cocamidopropylbetaine and benthiavalicarbisopropyl; cocamidopropylbetaine and cyazofamide; —cocamidopropylbetaine and R isomer IR5885; cocamidopropylbetaine, IR5885 and K₂HPO₃— KH₂PO₃; cocamidopropylbetaine, IR5885 and Fosetyl aluminium; glycinebetaine, IR5885 and Fosetyl aluminium; cocamidopropylbetaine, R isomer IR5885 and Fosetyl aluminium; glycinebetaine, R isomer IR5885 and Fosetyl aluminium; cocamidopropylbetaine, R isomer IR5885 and K₂HPO₃—KH₂PO₃; —glycinebetaine, K₂HPO₃—KH₂PO₃ and IR5885; —glycine betaine, K₂HPO₃— KH₂PO₃ and iprovalicarb;
glycinebetaine, K₂HPO₃— KH₂PO₃ and benthiavalicarb-isopropyl; glycinebetaine, K₂HPO₃—KH₂PO₃ and cyazofamide; —glycinebetaine, K₂HfPO₃— KH₂PO₃ and R isomer IR5885; cocamidopropylbetaine and ASA₂Cu; cocamidopropylbetaine and SA₂Cu; cocamidopropylbetaine and SACu, carnitine and K₂HPO₃, —carnitine and KH₂PO₃;
carnitine and K₂HPO₃— KH₂PO₃ and IR5885.

36. The composition according to claim 32, wherein the concentration of active principle ranges from 1% to 90%, preferably from 5% to 50%.

37. Use of the composition according to claim 32, for the control of phytopathogen fungi and bacteria and/or the mitigation of abiotic and biotic stress.

38. Use of the composition according to claim 32 for the stimulation of the natural defense systems of plants from abiotic or biotic stress and the induction of resistance in the plants themselves.

39. Use according to claim 37, wherein the application of the composition is effected on all parts of the plant, on the leaves, stems, branches and roots, or on the seeds themselves before being planted, or on the ground in which the plant grows.
